(19) United States
(12) Patent Application Publication
(10) Pub. No.: US 2022/0125882 A1
(43) Pub. Date: Apr. 28, 2022

(54) METHODS AND COMPOSITIONS FOR TREATMENT OF CANCER

(71) Applicant: Enzychem Lifesciences Corporation, Seoul (KR)

(72) Inventors: Ki-Young Sohn, Seoul (KR); Jae Wha Kim, Daejeon (KR); Sun Young Yoon, Seoul (KR)

(21) Appl. No.: 17/423,343

(22) PCT Filed: Jan. 16, 2019
(86) PCT No.: PCT/IB2019/000062

§ 371 Date: Jul. 15, 2021

(51) Int. Cl. A61K 38/19; A61K 31/231; A61K 31/675; A61K 31/704; A61K 31/7048; A61K 33/243; A61K 31/7068; A61K 31/138; A61K 31/10; A61P 35/00

(52) U.S. Cl. CPC A61K 38/193; A61K 31/231; A61K 31/675; A61K 31/704; A61P 35/00; A61K 33/243; A61K 31/7068; A61K 31/138; A61K 31/10; A61K 31/7048

(57) ABSTRACT

In one aspect, methods and compositions are provided for treating a neoplasia such as a solid tumor, the methods and composition comprising a) a granulocyte colony-stimulating factor (G-CSF) compound; and b) a monoacetyl diacylglycerol compound such as 1-palmitoyl-2-linoleoyl-3-acetylglycerol (PLAG).

### REFERENCE TO A “SEQUENCE LISTING,” A TABLE, OR A COMPUTER PROGRAM LISTING APPENDIX SUBMITTED AS AN ASCII FILE

The Sequence Listing written in file 055312-578F01WO_SL.txt, created Jan. 16, 2019, 1,703 bytes, machine format IBM-PC, MS Windows operating system, is hereby incorporated by reference.

### FIELD

In one aspect, methods and compositions are provided for treating a neoplasia such as a tumor, the methods and compositions comprising a) a granulocyte colony-stimulating factor (G-CSF) compound and b) a monoacetyl diacylglycerol compound such as 1-palmitoyl-2-linoleoyl-3-acetylglycerol (PLAG).

### BACKGROUND

Cancers are characterized by abnormal and uncontrolled cell growth. Cancer can involve any tissue in the body, and can spread outside the tissue of origin. Uncontrolled proliferation and other cellular abnormalities can lead to the formation of cancerous tumors. Tumors can disrupt the function of and destroy the tissues in which they originate, and, when cancer cells metastasize, secondary tumors can develop near to or disparate from the site of primary growth. Causes of cancer have been linked to various chemicals, viruses, bacteria, and environmental exposures.

Current cancer therapies such as radiation treatment and various chemotherapeutics have posed substantial toxicities and other side effects. Among others, use of granulocyte colony-stimulating factor (G-CSF) has resulted in undesired growth of cancerous tumors that are being targeted for removal. See, for instance, Voloshin et al., Blood, 118(12): 3426-3435 (2011).

It thus would be desirable to have improved cancer therapies.

### SUMMARY

In one aspect, we now provide new therapies for treatment and prevention of a patient suffering from cancer.

In particular aspects, methods and compositions are provided for reducing or suppressing tumor growth in a patient, including in patients that have receive or will receive a granulocyte colony-stimulating factor (G-CSF) compound.

In one aspect, the present methods comprise administering to a subject such as a human having a tumor or other neoplasia a therapeutically effective amount of:

a) a granulocyte colony-stimulating factor (G-CSF) compound; and

b) a monoacetyl diacylglycerol compound of Formula (I):

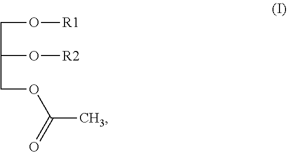

wherein R1 and R2 are independently a fatty acid group comprising 14 to 20 carbon atoms. The a) G-CSF compound and b) monoacetyl diacylglycerol compound of Formula (I) are suitably administered to the patient in combination or other coordinated manner.

In preferred aspects, the b) monoacetyl diacylglycerol is a compound of Formula II:

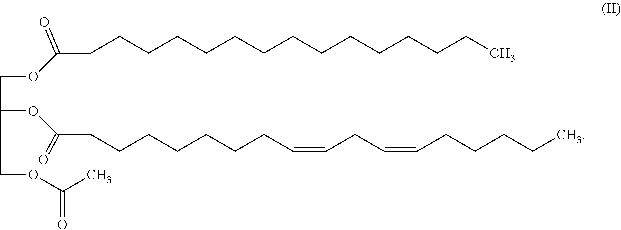 [SMILES: CCCCC/C=C\C/C=C\CCCCCCCC(=O)OC(COC(C)=O)COC(=O)CCCCCCCCCCCCCCC]

The compound of Formula (II) is also referred to as PLAG (1-palmitoyl-2-linoleoyl-3-acetylglycerol) or EC-18.

Significantly, we have now found that use of a monoacetyl diacylglycerol compound such as PLAG can reduce cancer tumor volume. Such tumor volume cancer reduction can occur while a patient is receiving treatment with a G-CSF compound, and contrasts to tumor volume increase that may occur with treatment with a G-CSF compound in the absence of combined administration with a monoacetyl diacylglycerol compound. See the results of Example 1, which follows.

In further aspects, the subject is also administered c) an additional chemotherapeutic agent distinct from the a) G-CSF compound and the b) monoacetyl diacylglycerol compound of Formula (I). For instance, the distinct chemotherapeutic agent may be cyclophosphamide, doxorubicin, etoposide, ifosfamide, mesna, cisplatin, gemcitabine and/or tamoxifen, or one or more other chemotherapeutic agents.

In certain aspects, prior to coordinated administration of a monoacetyl diacylglycerol compound of Formula (I) together with a G-CSF compound, the subject will have been previously treated with a G-CSF compound but without a monoacetyl diacylglycerol compound of Formula (I). For example, a patient may have been receiving G-CSF treatment in conjunction with a chemotherapeutic regime in the absence of administration of a monoacetyl diacylglycerol compound. After 1, 2, 3, 4, 5, 6, or 7 days, or 2, 3 or 4 weeks or more of such G-CSF therapy, a monoacetyl diacylglycerol compound of Formula (I) such as PLAG may be administered to the patient in coordination with continued G-CSF administration.

In a further aspect, pharmaceutical compositions are provided comprising a) a granulocyte colony-stimulating factor (G-CSF) compound and b) a monoacetyl diacylglycerol compound such as PLAG (1-palmitoyl-2-linoleoyl-3-acetylglycerol). Preferred pharmaceutical compositions are suitable for treating cancer including solid tumors in a subject.

In a yet further aspect, kits are provided for use to treat or prevent a neoplasia including a solid tumor. Kits of the invention suitably may comprise a) a granulocyte colony-stimulating factor (G-CSF) compound; and b) a monoacetyl diacylglycerol compound such as PLAG (1-palmitoyl-2-linoleoyl-3-acetylglycerol). Preferably, a kit will comprise a therapeutically effective amount of each of a G-CSF compound and a monoacetyl diacylglycerol compound such as PLAG. Preferred kits also may comprise instructions for use of the PLAG and G-CSF compound to treat a cancer such as a solid tumor. The instructions suitably may be in written form, including as a product label.

### BRIEF DESCRIPTION OF THE DRAWINGS

FIG. 1A is an experimental scheme in order to evaluate the effect of EC-18 in combination with G-CSF in tumor-bearing mice. The control group was PBS-treated (4 mice, Control), and experimental groups were treated with G-CSF administration group (5 mice, PEG-G-CSF), gemcitabine administration group (5 mice, Gemcitabine), G-CSF and gemcitabine co-administration group (5 mice, Gem+PEG), and EC-18 co-administration group (5 mice, Gem+PEG+EC-18).

FIG. 1B shows changes in body weights, tumor weights, ratios of the tumor weights to the body weights, and the spleen weights of the mice in the experiment of FIG. 1A.

FIG. 1C shows a photograph of tumors from the tumor-bearing mice in the experiment of FIG. 1A.

FIG. 2A shows the tumor sizes from the tumor-bearing mice in the experiment of FIG. 1A.

FIG. 2B is a table showing numerical values of the graph in FIG. 2A.

FIGS. 3A-3B show inhibition of abnormal metastasis of breast cancer cells in TAN ((Tumor associated Neutrophil) and G-CSF co-culture by PLAG treatment. FIG. 3A shows that in the TAN and G-CSF co-culture environment, the mobility reduction in breast cancer cell was effective with PLAG treatment. Green fluorescence was expressed in cytoskeleton, and nucleus was stained with PI. FIG. 3B shows that transwell invasion assay was used to confirm the abnormal invasion inhibition of cancer cells by PLAG treatment in TAN and G-CSF co-culture environments.

FIGS. 4A-4D show changes in epithelial-mesenchymal transition (EMT) marker expression of breast cancer cell in TAN and G-CSF co-culture by PLAG treatment. FIG. 4A shows that a gel separation method was used to verify the expression of EMT marker gene. FIGS. 4B-4D show quantitation of the band intensity of the target genes (FIG. 4B: snail/actin; FIG. 4C: vimentin/actin; and FIG. 4D: NCAD/actin) using Image J.

FIGS. 5A-5B show changes in cytokine expression of breast cancer cell in TAN and G-CSF co-culture by PLAG Treatment. FIG. 5A show that the secretion of TGF-β, which is an abnormal transcriptional activator of cancer cell, was confirmed by ELISA. FIG. 5B shows that the secretion level of IFN-γ, a cancer cell activation inhibitor, was quantitatively verified by ELISA.

FIG. 6A-6B show changes in TGF-β signaling pathway by PLAG treatments. FIG. 6A shows that the degree of Smad proteins activity by PLAG treatment was confirmed by Western blotting. FIG. 6B shows that the change of complex formation of Smads by PLAG treatment was confirmed by IP method.

FIG. 7 shows Smad2/3 translocation change by PLAG treatment. The nuclear localization of Smad2/3 protein by PLAG treatment was qualitatively verified using confocal.

### DETAILED DESCRIPTION

The terms PLAG, EC-18 and 1-palmitoyl-2-linoleoyl-3-acetylglycerol are used interchangeably herein and designate the same compound herein.

### Definitions

The abbreviations used herein have their conventional meaning within the chemical and biological arts. The chemical structures and formulae set forth herein are constructed according to the standard rules of chemical valency known in the chemical arts.

It will be apparent to one skilled in the art that certain compounds disclosed herein may exist in tautomeric forms, all such tautomeric forms of the compounds being within the scope of the invention.

The terms “a” or “an,” as used in herein means one or more. For example, the singular forms “a,” “an” and “the” are intended to include the plural forms as well, unless the context clearly indicates otherwise. It will be further understood that the terms “comprise”, “include”, “have”, etc. when used in this specification, specify the presence of stated features, regions, integers, steps, processes, operations, elements and/or components but do not preclude the presence or addition of one or more other features, regions, integers, steps, processes, operations, elements, components, and/or combinations thereof.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. The term “treating” and conjugations thereof, may include prevention of an injury, pathology, condition, or disease. In embodiments, treating is preventing. In embodiments, treating does not include preventing.

The term “prevent” refers to a decrease in the occurrence of disease symptoms in a patient. As indicated above, the prevention may be complete (e.g., no detectable symptoms) or partial, such that fewer symptoms are observed than would likely occur absent treatment.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient or subject is human.

An “effective amount” or a “therapeutically effective amount” is an amount sufficient for a compound to accomplish a stated purpose relative to the absence of the compound (e.g. achieve the effect for which it is administered, treat a disease, reduce enzyme activity, increase enzyme activity, reduce a catabolic enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A first therapy (e.g. administration of either i) a G-CSF compound or ii) a monoacetyl diacylglycerol compound of Formula (I) such as PLAG) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours or up to about one 1 week before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours or up to about one 1 week after) the administration of a second therapy (e.g. administration of either i) a monoacetyl diacylglycerol compound of Formula (I) such as PLAG or ii) a G-CSF compound) to a subject which had, has, or is susceptible to cancer, including a subject that has been diagnosed with a solid tumor. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The terms “proliferative disorder” and “proliferative disease” refer to disorders associated with abnormal cell proliferation such as cancer.

“Tumor” and “neoplasm” or similar term as used herein refer to any mass of tissue that result from excessive cell growth or proliferation, either benign or malignant including pre-cancerous lesions.

As discussed, the present methods and compositions comprise a) a granulocyte colony-stimulating factor (G-CSF) compound; and b) a monoacetyl diacylglycerol compound of Formula (I) such as PLAG.

The present methods and compositions can effectively reduce or suppress tumor growth in a patient, for example a cancer patient that receives a therapy of a) a granulocyte colony-stimulating factor (G-CSF) compound and b) a monoacetyl diacylglycerol compound of Formula (I) such as PLAG. Co-treatment with G-CSF and PLAG may result in a 5, 10, 15, 20, 25, 30, 35, 40, 45 or 50 percent or more reduction in tumor volume.

A wide variety of type of cancers may be treated in accordance with the present methods and compositions. For instance, a cancer to be treated may be a solid tumor. Illustrative cancers for which the invention can be used include, but are not limited to bladder cancer, leukemias (e.g., kemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, gastric and esophageal cancer, head and neck cancer, rectal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

Granulocyte-colony stimulating factor (G-CSF), also known as colony-stimulating factor 3 (CSF 3), is a glycoprotein that stimulates the hematopoietic precursor cells in the bone marrow to proliferate and differentiate into mature granulocytes and stem cells and release them into the bloodstream. In humans, it exists in two active forms, the more abundant of which is 174 amino acids long; the other is 177 amino acids long. The pharmaceutical analogs of naturally occurring G-CSF are recombinant forms of the human 174-amino acid peptide (rhG-CSF), and include: filgrastim (e.g. Neupogen® from Amgen), which made in E. coli, having the same activity, but differing from the natural glycoprotein in having an N-terminal methionine residue and lacking glycosylation; lenograstim (e.g., Granocyte® from Chugai), which is made in mammalian cells (Chinese Hamster Ovary (CHO) cells), and so is essentially indistinguishable from human G-CSF; pegfilgrastim, a PEGylated form of filgrastim, (e.g., Neulasta® from Amgen and Neulastim® from Roche), having a 20 kD monomethoxypolyethylene glycol moiety covalently bound to the N-terminal methionyl residue of filgrastim, which increases solubility and duration of action compared to filgrastim.

Granulocyte-colony stimulating factor (G-CSF) as referred to herein includes without limitation all of the above forms.

A chemical synthetic method for the preparation of monoacetyldiacylglycerol compounds of Formula (I) is shown, for example, in Korean Registered Patents No. 10-0789323 and No. 10-1278874, the contents of which are incorporated herein by reference. For example, PLAG can be synthesized by acylating the hydroxy groups of glycerol with acetyl, palmitoyl and linoleoyl functional groups.

Therapeutically effective amounts of a monoacetyl diacylglycerol compound of Formula (I) such as PLAG and a granulocyte colony-stimulating factor (G-CSF) compound can be initially determined from cell culture assays. Target concentrations will be those concentrations of active compound(s) that are capable of achieving the methods described herein, as measured using the methods described herein or known in the art. Treatment amounts of a monoacetyl diacylglycerol compound of Formula (I) such as PLAG and a granulocyte colony-stimulating factor (G-CSF) compound also have been previously reported.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The frequency of administration of the composition of the present invention is not particularly limited, but it may be administered once a day or several times a day with divided dosage.

A monoacetyl diacylglycerol compound of Formula (I) such as PLAG and a granulocyte colony-stimulating factor (G-CSF) compound can be administered to a subject by any of a number of routes such as topical contact, oral, intravenous, intraperitoneal, intramuscular, intralesional, intrathecal, intranasal or subcutaneous administration, or the implantation of a slow-release device, e.g., a mini-osmotic pump, to a subject. Parenteral administration includes, e.g., intravenous, intramuscular, intra-arteriole, intradermal, subcutaneous, intraperitoneal, intraventricular, and intracranial.

Pharmaceutical compositions may include compositions wherein one or both of a monoacetyl diacylglycerol compound of Formula (I) such as PLAG and a granulocyte colony-stimulating factor (G-CSF) compound the PLAG compound is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

Pharmaceutical composition may be manufactured with additional pharmaceutically acceptable carrier for each formulation. As used herein, the term “pharmaceutically acceptable carrier” may refer to a carrier or diluent that does not stimulate organism and not inhibiting biological activity and characteristic of the injected compound. The type of the carrier that can be used in the present invention is not particularly limited, any carrier conventionally used in the area of industry and pharmaceutically acceptable may be used.

Saline, sterilized water, IV fluids, buffer saline, albumin injection solution, dextrose solution, maltodextrin solution, glycerol, ethanol are non-limiting examples of the usable carriers. These carriers may be used alone or in combination of two or more. The carrier may include a non-naturally occurring carrier. If necessary, other conventionally used additives like an antioxidant, a buffer and/or a bacteriostatic agent may be added and used. It may be formulated with diluent, a dispersant, a surfactant, a bonding agent, a lubricant to make an injection solution like aqueous solution, suspension, emulsion, and pills, capsules, granules or tablets, and the like.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

As discussed, kits are also provided. For instance, in this aspect, a monoacetyl diacylglycerol compound of Formula (I) such as PLAG and a granulocyte colony-stimulating factor (G-CSF) compound a PLAG compound each suitably can be packaged in suitable containers labeled for a specified treatment. The containers can include a PLAG compound or composition and a granulocyte colony-stimulating factor (G-CSF) compound and one or more of a suitable stabilizer, carrier molecule and/or the like, as appropriate for the intended use. In other embodiments, the kit further comprises one or more therapeutic reagents for an intended treatment, such as one or more additional chemotherapeutic agents. A product can include a container (e.g., a vial, jar, bottle, bag, or the like) containing a PLAG compound or composition and/or a granulocyte colony-stimulating factor (G-CSF) compound. In addition, an article of manufacture or kit further may include, for example, packaging materials, instructions for use, syringes, delivery devices, for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

As discussed, a granulocyte colony-stimulating factor (G-CSF) compound and a monoacetyl diacylglycerol compound such as PLAG can be administered in combination with an anti-neoplasia such as a chemotherapeutic agent, e.g. one or more of alkylating agents (e.g., platinum-based drugs, tetrazines, aziridines, nitrosoureas, nitrogen mustards), anti-metabolites (e.g., anti-folates, fluoropyrimidines, deoxynucleoside analogues, thiopurines), anti-microtubule agents (e.g., vinca alkaloids, taxanes), topoisomerase inhibitors (e.g., topoisomerase I and II inhibitors), cytotoxic antibiotics (e.g., anthracyclines) and immunomodulatory drugs (e.g., thalidomide and analogs). In certain aspects, the chemotherapeutic agent may be one or more of cyclophosphamide, doxorubicin, etoposide, ifosfamide, mesna, cisplatin, gemcitabine and/or tamoxifen.

A granulocyte colony-stimulating factor (G-CSF) compound and a monoacetyl diacylglycerol compound such as PLAG suitably are administered in a coordinated manner, for example either simultaneously or sequentially. For instance, a granulocyte colony-stimulating factor (G-CSF) compound and a monoacetyl diacylglycerol compound such as PLAG may be administered to a subject at substantially the same time, or the agents instead may be administered to the subject at different times, suitably within hours although longer periods between the separate administrations also may be suitable.

### EXAMPLES

Although the foregoing section has been described in some detail by way of illustration and example for purposes of clarity of understanding, it is apparent to those skilled in the art that certain minor changes and modifications will be practiced in light of the above teaching. Therefore, the description and examples should not be construed as limiting the scope of any invention described herein.

All references cited herein, including patent applications and publications, are hereby incorporated by reference in their entirety.

### Example 1: Anti-Cancer Effects of EC-18 in Combination with G-CSF on Human Myeloma Cell in a Xenograft Mouse Model

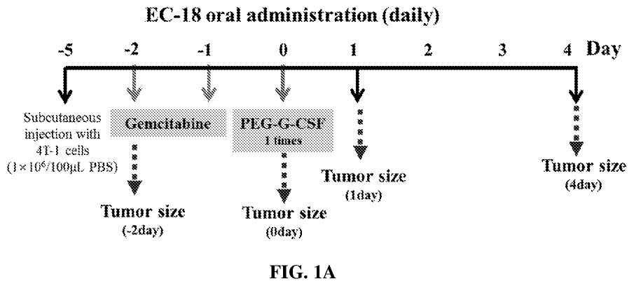

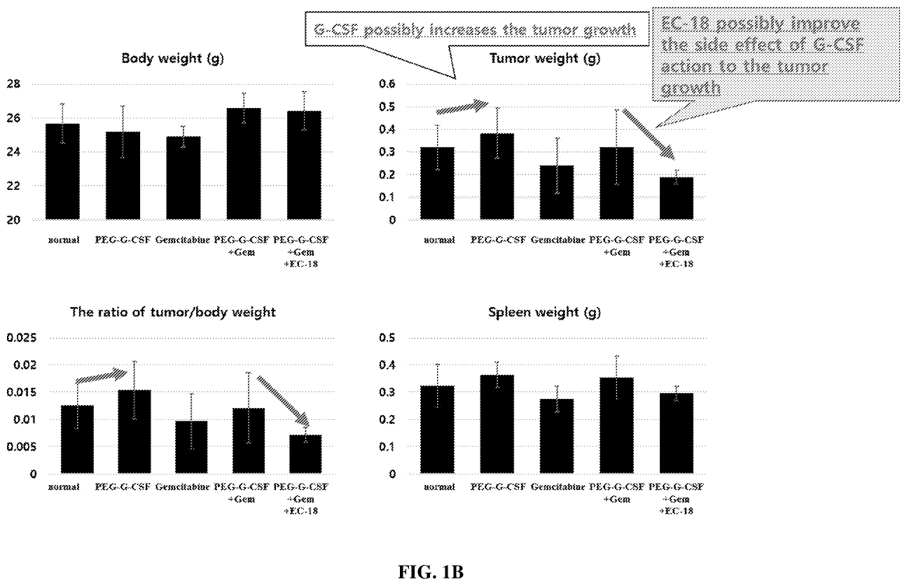

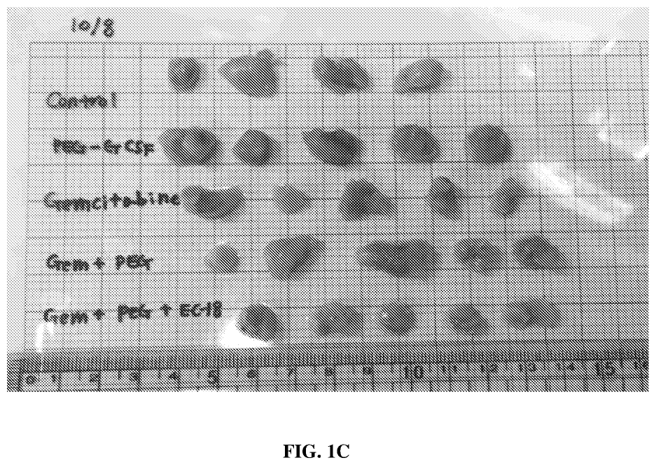

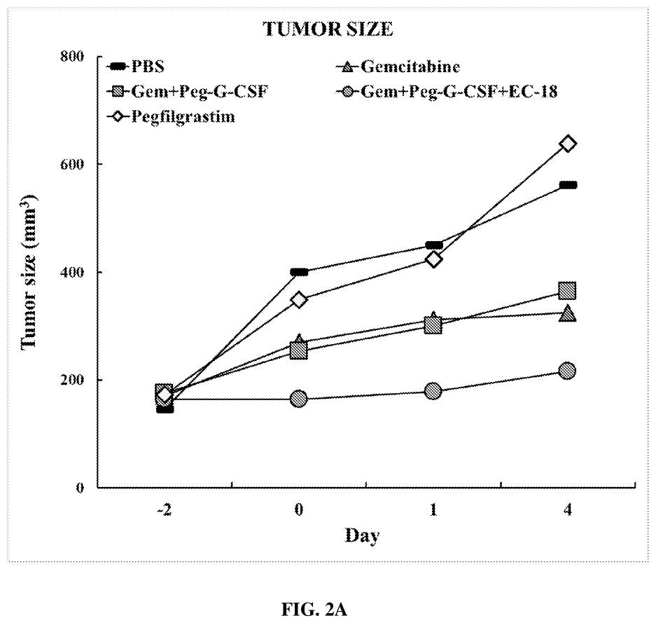

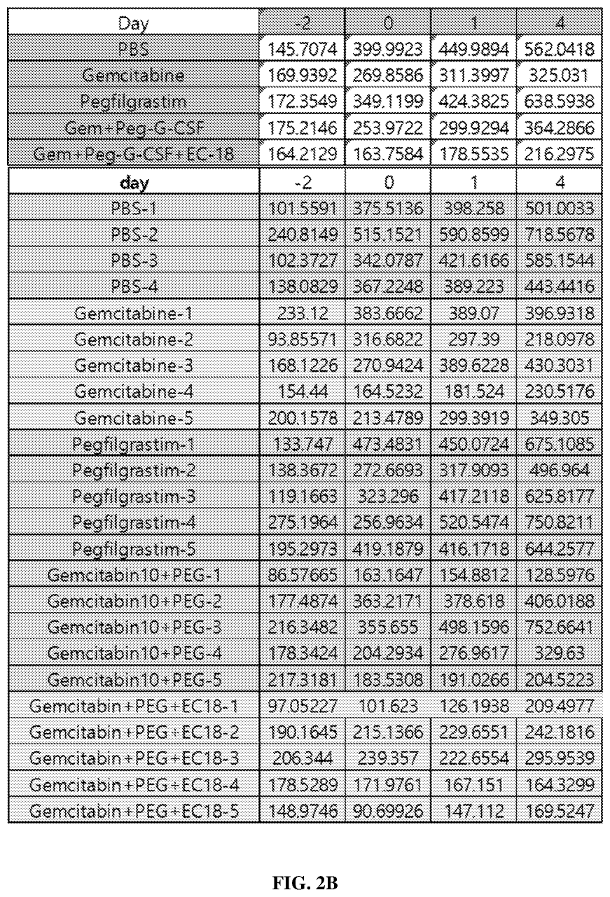

To demonstrate that administration of the compound of Formula 2 (EC-18) can overcome the increased tumor growth induced by administration of G-CSF, tumor-bearing mice were prepared by administrating by subcutaneous injection 1×10⁶ cells/100 μL phosphate buffered saline (PBS) of 4T1 cell line into BALB/c 7w mice (Day 0). On Day 2 and Day 3, the mice were administered 10 mg/kg of gemcitabine twice. On Day 4, 3 μg/mouse of PEG-G-CSF (Pegfilgrastim) was administrated via subcutaneous injection to the mice once. On every day of the study, 50 mg/kg of EC-18 diluted with PBS was administrated orally to the mice. A total of 24 tumor-bearing mice were classified into 5 groups consisting of control group (4 mice, Control), G-CSF administration group (5 mice, PEG-G-CSF), gemcitabine administration group (5 mice, Gemcitabine), G-CSF and gemcitabine co-administration group (5 mice, Gem+PEG), and EC-18 co-administration group (5 mice, Gem+PEG+EC-18), as summarized in Table 1 and FIG. 1A.

TABLE 1
Mice Administration Groups.

| Group | n | Gemcitabine | PEG-G-CSF | EC-18 |
|---|---|---|---|---|
| Control | 4 | — | —— | — |
| PEG-G-CSF | 5 | | 3 μg | — |
| Gemcitabine | 5 | 10 mg/kg | — | — |
| Gem + PEG | 5 | 10 mg/kg | 3 μg | — |
| Gem + PEG + EC-18 | 5 | 10 mg/kg | 3 μg | 50 mg/kg |

On Day 8, all mice were sacrificed and their tumors removed. The size and weight of the tumors were measured. The results are shown in Tables 2 and 3, and photographs of the tumors are shown in FIG. 1C.

TABLE 2
Tumor Weight

| | Tumor Weight (×0.01 g) | | | | | | |
|---|---|---|---|---|---|---|---|
| | No. 1 | No. 2 | No. 3 | No. 4 | No. 5 | Average | Increase |
| Control | 19 | 41 | 38 | 30 | — | 32 ± 10 | — |
| PEG-G-CSF | 47 | 30 | 53 | 30 | 31 | 38 ± 11 | +19.4 |
| Gemcitabine | 38 | 9 | 32 | 27 | 14 | 24 ± 12 | −25.0 |
| Gem + PEG | 11 | 41 | 54 | 23 | 31 | 32 ± 16 | +0.0 |
| Gem + PEG + EC18 | 20 | 22 | 15 | 16 | 21 | 19 ± 3 | −41.2 |

TABLE 3
% Increase (+) or % Decrease (−) of Tumor Weight

| | % weight | | | | | | |
|---|---|---|---|---|---|---|---|
| | No. 1 | No. 2 | No. 3 | No. 4 | No. 5 | Average | Increase |
| Control | 0.73 | 1.71 | 1.43 | 1.15 | — | 1.25 ± 0.42 | — |
| PEG-G-CSF | 2.02 | 1.12 | 2.20 | 1.20 | 1.17 | 1.54 ± 0.52 | +22.7 |
| Gemcitabine | 1.55 | 0.36 | 1.32 | 1.08 | 0.54 | 0.97 ± 0.51 | −22.6 |
| Gem + PEG | 0.41 | 1.47 | 2.13 | 0.87 | 1.17 | 1.21 ± 0.65 | −3.5 |
| Gem + PEG + | 0.80 | 0.83 | 1 0.56 | 0.57 | 0.81 | 0.72 ± 0.14 | −43.0 |
| EC-18 | | | | | | | |

The data show that the tumor weight in gemcitabine-treated mice decreased by about 25% compared to control, demonstrating the anti-cancer effects of treatment with gemcitabine. The data show that the tumor weight in mice administered G-CSF (Filgrastitn), however, increased by about 19.4% compared to control, exhibiting the side effect of promoting tumor growth by G-CSF. The data demonstrate that, in mice co-administered gemcitabine and G-CSF, no change of tumor weight was observed compared to the control, implicating the efficacy of chemotherapy is reduced by co-administration of G-CSF regardless of any therapeutic effect on neutropenia.

The data demonstrate that co-administration of EC-18 with gemcitabine and G-CSF significantly decreased tumor weight by up to 41%, which is more of a reduction than the reduction achieved by administration of gemcitabine only. The result is consistent with the percent change of tumor weight over the body weight of tumor-bearing mice (Table 3), and photographs of tumor tissue (FIG. 1C). The data demonstrate that EC-18 (i.e., PLAG or the compound of Formula 2 of the present application) minimizes the side effect of G-CSF on tumor growth.

### Example 2: Inhibition of Abnormal Metastasis of MDA-MB-231 Breast Cancer Cells in TAN Environment and G-CSF Co-Stimulation by PLAG Treatment

### Materials and Methods

### Cell Culture

MDA-MB-231 and HL60 cells were obtained from the American Type Culture Collection (ATCC, Rockville, Md., USA). MDA-MB-231 cells were grown in DMEM medium (WELGENE, Seoul, Korea) containing 10% fetal bovine serum (HyClone, Waltham, Mass., USA), 1% antibiotics (100 mg/l streptomycin, 100 U/ml penicillin), and 0.4% 2-Mercaptoethanol (Sigma Aldrich, St. Louis, Mo., USA). HL60 cells were grown in DMEM medium containing 10% fetal bovine serum and 1% antibiotics (100 mg/l streptomycin, 100 U/ml penicillin). Cells were grown at 37° C. in a 5% CO2 atmosphere. To differentiate HL60 cells into neutrophil-like cells, cells were grown in medium with 10% DMSO (Sigma Aldrich) for 5 days.

### Wound Healing Assay

MDA-MB-231 were seeded in 12 well plate with cover glass, and incubated to 100% confluence at well. After the incubation, make the wound cell monolayer on center of well. A treated PLAG and neutrophil and G-CSF co-stimulation for indicate time. After treatment the indicated time and dose at 37° C. in a 5% CO2 atmosphere, the cells were fixed with 3.7% formaldehyde for 20 min and permeabilized with 0.2% Triton X-100 for 20 min and stained with 0.1% Phalloidin-FITC for 40 min. Fluorescence was detected by confocal (Carl Zeiss, Thornwood, N.Y., USA).

### Confocal (Immunofluorescence)

MDA-MB-231 were seeded in 12 well plate with cover glass, and incubated to 60% confluence at well. After the incubation, treated PLAG and neutrophil and G-CSF co-stimulation for indicate time. After treatment the indicated time at 37° C. in a 5% CO2 atmosphere, the cells was fixed with 3.7% formaldehyde for 20 min and permeabilized with 0.2% Triton X-100 for 20 min and stained with 0.1% Phalloidin-FITC for 40 min. Cells were washed with PBS twice and reacted with specific anti-body for over-night at 4° C. Cells were washed with PBS twice and reacted secondary antibody. For nucleus detection, it stained with 1% Hoechst33342 for 20 min. Fluorescence was detected by confocal (Carl Zeiss, Thornwood, N.Y., USA).

### Transwell Invasion Assay

Quantitative macrophage chemoattraction assays were performed using a modified Boyden chamber (SPL lifescience, Seoul, Korea) with 8.0-μm pore polycarbonate membrane inserts with matrigel-coated in 24-well plates. The lower chamber was filled with apoptotic neutrophils for chemoattraction. The MDA-MB-231 cells (5×10⁴ cells/ml) in serum-free medium were added into the upper chamber and treated with PLAG. The cells were allowed to chemoattraction (Neutrophil co-culture supernatant) for indicate times at 37° C. in a 5% CO2 atmosphere. The non-chemoattracted cells were removed from the upper surface of the membrane by scraping with a cotton swab, and the chemoattracted cells were calculate by MTT assay.

### Results

Using an ex vivo environment using transwell was confirmed the effect of PLAG to identify MDA-MB-231 abnormal metastasis inhibitory effects in breast cancer cells by the TAN (Tumor associated Neutrophil) environment. As a result, it was confirmed that mobility of neutrophil-stimulated MDA-MB-231 cells was induced, while mobility decreased by PLAG treatment. Also, we confirmed the transwell invasion effect of MDA-MB-231 breast cancer cells using culture sup., indicating that invasion is increased in neutrophil-stimulated culture sup., whereas invasion of cancer cells is decreased in PLAG treatment group. On the other hand, G-CSF and neutrophil co-stimulation increased the metastasis of cancer cells more than neutrophil only group. In the co-stimulation group, the transwell invasion, as well as the mobility of the cancer cells, was further increased, whereas the PLAG-treated group inhibited the cancer cell abnormal-metastasis in the same manner as the neutrophil-stimulation group.

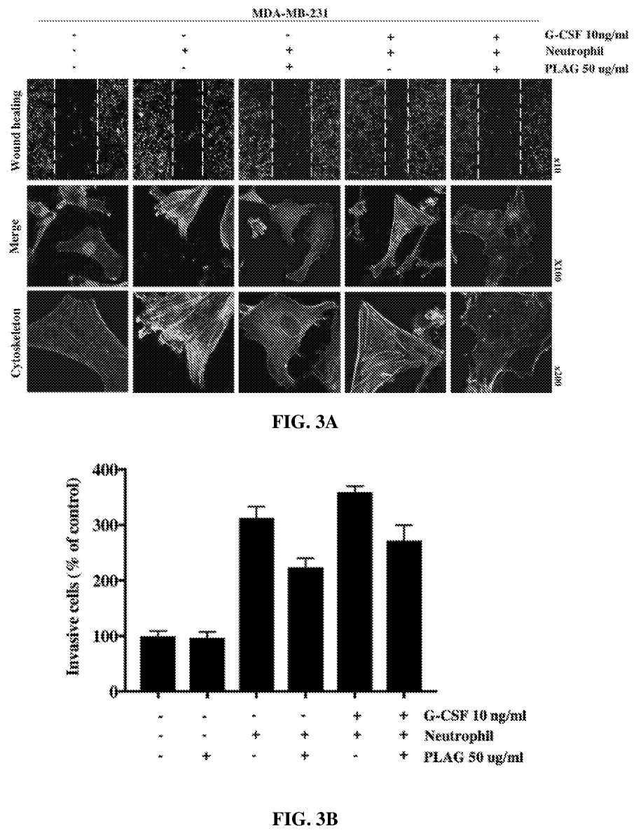

TABLE 4
Raw data for FIG. 3B. Invasive cell count (MTT assay, % transform)

| | | | | | | Neutrophil + |
|---|---|---|---|---|---|---|
| | | | Neutrophil | Neutrophil + | Neutrophil + | G-CSF + |
| | None | PLAG | only | PLAG | G-CSF | PLAG |
| Sample 1 | 106.250 | 89.583 | 327.083 | 212.500 | 366.667 | 252.083 |
| Sample 2 | 93.750 | 104.167 | 297.917 | 235.417 | 352.083 | 383.333 |
| Sample 3 | 85.417 | 102.083 | 331.250 | 216.667 | 383.333 | 287.500 |

### Example 3: Inhibition of EMT Marker Expression in TAN Environment and G-CSF Co-Stimulation by PLAG Treatment

### Materials and Methods

### Cell Culture

MDA-MB-231 and HL60 cells were obtained from the American Type Culture Collection (ATCC, Rockville, Md., USA). MDA-MB-231 cells were grown in DMEM medium (WELGENE, Seoul, Korea) containing 10% fetal bovine serum (HyClone, Waltham, Mass., USA), 1% antibiotics (100 mg/l streptomycin, 100 U/ml penicillin), and 0.4% 2-Mercaptoethanol (Sigma Aldrich, St. Louis, Mo., USA). HL60 cells were grown in DMEM medium containing 10% fetal bovine serum and 1% antibiotics (100 mg/l streptomycin, 100 U/ml penicillin). Cells were grown at 37° C. in a 5% CO2 atmosphere. To differentiate HL60 cells into neutrophil-like cells, cells were grown in medium with 10% DMSO (Sigma Aldrich) for 5 days.

### Polymerase Chain Reaction (PCR)

Total RNA was extracted using RiboEx (GeneAll biotechnology, Seoul, Korea) according to the manufacturer's instructions, and cDNA was generated using ReverseAids cDNA synthesis kit (Thermo Scientific, Waltham, Mass., USA) according to the manufacturer's instructions. PCR was performed with the following temperature profile: a pre-denaturation step of 10 min at 95° C., followed by 35 cycles of 95° C. for 30 sec, annealing temperature for 30 sec and 72° C. for 30 sec and a final exposure to 72° C. for 10 min. Specific primer sequence for amplification was Table 5.

TABLE 5
Primer sequence for the amplification of target gene

| | | Amplification size | Annealing Temp. |
|---|---|---|---|
| Gene | Primer Sequence (5′-3′) | (bp) | (° C.) |
| N-cadherin | For: GACAATGCCCCTCAAGTGTT | 179 | 59.5 |
| | (SEQ ID NO: 1) | | |
| | Rev: CCATTAAGCCGAGTGATGGT | | |
| | (SEQ ID NO: 2) | | |
| Vimentin | For: GAGAACTTTGCCGTTGAAG | 170 | 59.5 |
| | (SEQ ID NO: 3) | | |
| | Rev: TCCAGCAGCTTCCTGTAGGT | | |
| | (SEQ ID NO: 4) | | |
| Snail | For: CCCCAATCGGAAGCCTAACT | 157 | 60.0 |
| | (SEQ ID NO: 5) | | |
| | Rev: ACAGAGTCCCAGATGAGCA | | |
| | (SEQ ID NO: 5) | | |
| β-actin | For: CAAGGTCATCCATGACAACTTTG | 496 | 58.0 |
| | (SEQ ID NO: 7) | | |
| | Rev: GTCCACCACCCTGTTGCTGTAG | | |
| | (SEQ ID NO: 8) | | |

### Results

PCR was performed to confirm the suppression effect of MDA-MB-231 breast cancer cells on EMT maker expression by stimulation of TAN environment and G-CSF by PLAG treatment. As a result, expression levels of SNAIL and NCAD, Vimentin, which are EMT (Epithelial mesenchymal transition) makers, were increased in neutrophil and G-CSF co-stimulation group, whereas expression was decreased by PLAG treatment.

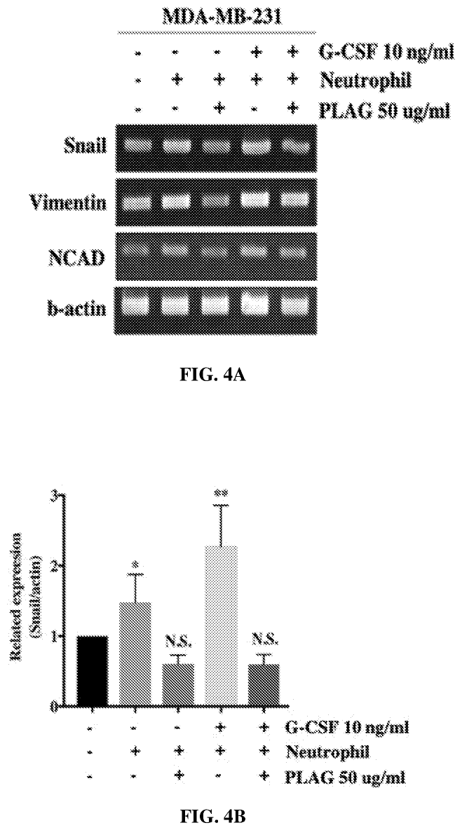

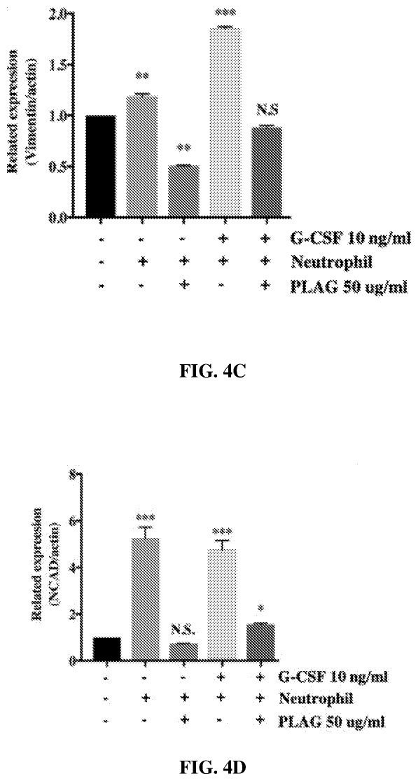

TABLE 6
Raw data for (FIGS. 4B-4D): PCR bend intensity (relative intensity)

| | | | | |
|---|---|---|---|---|
| | | | | Neutrophil + |
| | Neutrophil | Neutrophil + | Neutrophil + | G-CSF + |
| None | only | PLAG | G-CSF | PLAG |

Snail

| | | | | | |
|---|---|---|---|---|---|
| Sample 1 | 1 | 1.916 | 0.749 | 2.781 | 0.758 |
| Sample 2 | 1 | 1.134 | 0.527 | 1.635 | 0.525 |
| Sample 3 | 1 | 1.383 | 0.531 | 2.4 | 0.516 |

N-cadherin

| | | | | | |
|---|---|---|---|---|---|
| Sample 1 | 1 | 5.310 | 0.723 | 4.517 | 1.506 |
| Sample 2 | 1 | 4.730 | 0.747 | 4.511 | 1.584 |
| Sample 3 | 1 | 5.681 | 0.732 | 5.214 | 1.594 |

Vimentin

| | | | | | |
|---|---|---|---|---|---|
| Sample 1 | 1 | 1.16 | 0.501 | 1.832 | 0.46 |
| Sample 2 | 1 | 1.208 | 0.512 | 1.854 | 0.901 |
| Sample 3 | 1 | 1.192 | 0.504 | 1.867 | 0.885 |

### Example 4: Changes of Cytokine Secretion in TAN Environment and G-CSF Co-Stimulation by PLAG Treatment

Materials and Methods

Cell Culture

MDA-MB-231 and HL60 cells were obtained from the American Type Culture Collection (ATCC, Rockville, Md., USA). MDA-MB-231 cells were grown in DMEM medium (WELGENE, Seoul, Korea) containing 10% fetal bovine serum (HyClone, Waltham, Mass., USA), 1% antibiotics (100 mg/l streptomycin, 100 U/ml penicillin), and 0.4% 2-Mercaptoethanol (Sigma Aldrich, St. Louis, Mo., USA). HL60 cells were grown in DMEM medium containing 10% fetal bovine serum and 1% antibiotics (100 mg/l streptomycin, 100 U/ml penicillin). Cells were grown at 37° C. in a 5% CO2 atmosphere. To differentiate HL60 cells into neutrophil-like cells, cells were grown in medium with 10% DMSO (Sigma Aldrich) for 5 days.

ELISA

The levels of cytokine secretion in the cell supernatants or plasma were analyzed using an enzyme-linked immunosorbent assay (ELISA) specifically for cytokines from BD Bioscience according to the manufacturer's protocol. The absorbance was measured at 450 nm using an EMax Endpoint ELISA microplate reader (Molecular Devices Corporation, Sunnyvale, Calif., USA).

Results

The secretion level of TGF-β, which is a cancer cell metastasis activity marker by TAN environment and G-CSF co-stimulation, was confirmed by ELISA. As a result, the secretion level of TGF-β was increased in TAN and G-CSF costimulation environment. However, it was confirmed that expression of TGF-β did not significantly decrease upon treatment with PLAG.

The secretion of IFN-γ, an anti-cancer cytokine, was significantly increased by PLAG treatment, while it did not change the secretion of TGF-β. In contrast, the secretion of IFN-γ in the G-CSF co-stimulation group was significantly lower than that in the neutrophil only group.

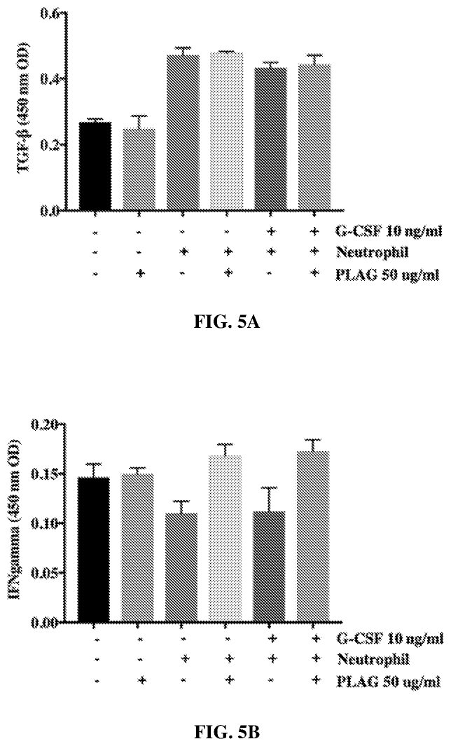

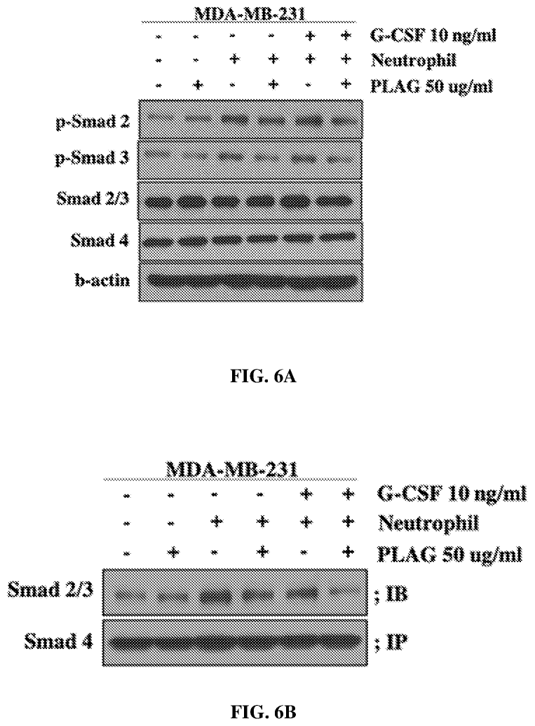

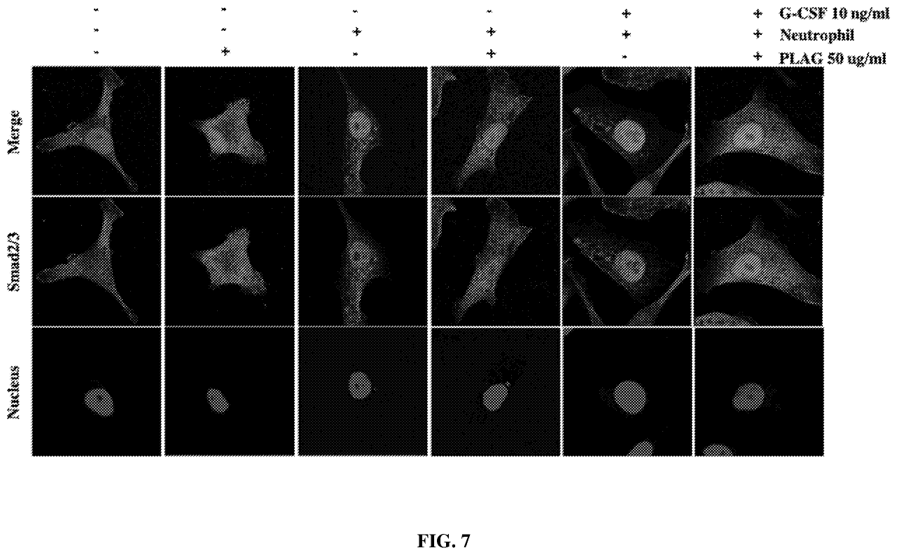

TABLE 7
Raw data for FIG. 5A from TGF-β ELISA (450 nm OD)

| | | | Neutrophil | Neutrophil + | Neutrophil + | Neutrophil + |
|---|---|---|---|---|---|---|
| | None | PLAG | only | PLAG | G-CSF | G-CSF + PLAG |
| Sample 1 | 0.279 | 0.248 | 0.447 | 0.484 | 0.439 | 0.45 |
| Sample 2 | 0.265 | 0.287 | 0.485 | 0.476 | 0.446 | 0.413 |
| Sample 3 | 0.262 | 0.208 | 0.484 | 0.474 | 0.414 | 0.468 |

TABLE 8
Raw data for FIG. 5B from IFN-γ ELISA (450 nm OD)

| | | | Neutrophil | Neutrophil + | Neutrophil + | Neutrophil + |
|---|---|---|---|---|---|---|
| | None | PLAG | only | PLAG | G-CSF | G-CSF + PLAG |
| Sample 1 | 0.131 | 0.144 | 0.124 | 0.181 | 0.139 | 0.161 |
| Sample 2 | 0.153 | 0.149 | 0.104 | 0.158 | 0.102 | 0.173 |
| Sample 3 | 0.155 | 0.156 | 0.102 | 0.164 | 0.094 | 0.184 |

### Example 5: Inhibition of TGF-β-Dependent Cancer Cell Metastasis Signal Pathway by PLAG Treatment

Materials and Methods

Cell Culture

MDA-MB-231 and HL60 cells were obtained from the American Type Culture Collection (ATCC, Rockville, Md., USA). MDA-MB-231 cells were grown in DMEM medium (WELGENE, Seoul, Korea) containing 10% fetal bovine serum (HyClone, Waltham, Mass., USA), 1% antibiotics (100 mg/l streptomycin, 100 U/ml penicillin), and 0.4% 2-Mercaptoethanol (Sigma Aldrich, St. Louis, Mo., USA). HL60 cells were grown in DMEM medium containing 10% fetal bovine serum and 1% antibiotics (100 mg/l streptomycin, 100 U/ml penicillin). Cells were grown at 37° C. in a 5% CO2 atmosphere. To differentiate HL60 cells into neutrophil-like cells, cells were grown in medium with 10% DMSO (Sigma Aldrich) for 5 days.

Immunoprecipitation (IP)

MDA-MB-231 cells treated with PLAG and stimulated to induce neutrophil and G-CSF co-stimulation for various times at 37° C. in a 5% CO2 atmosphere were lysed using ice-cold IP lysis buffer (25 mM Tris-HCl pH 7.4, 150 mM NaCl, 1% NP-40, 1 mM EDTA, 5% glycerol). Extracted proteins were incubated with Surebeads Protein G specific antibody bound magnetic beads (Bio-Rad, Hercules, Calif., USA). Beads were washed with PBS with Tween 20 (PBST) and target proteins eluted in 1× sample buffer and analyzed by Western blotting.

Confocal (Immunofluorescence)

MDA-MB-231 cells were seeded in 12 well plate with cover glass, and incubated to 60% confluence at well. After the incubation, treated PLAG and neutrophil and G-CSF co-stimulation for indicate time. After treatment the indicated time at 37° C. in a 5% CO2 atmosphere, the cells were fixed with 3.7% formaldehyde for 20 min and permeabilized with 0.2% Triton X-100 for 20 min for staining. Cells were washed with PBST twice and reacted with specific antibody (SMAD2/3) for over-night at 4° C. Cells were washed with PBST twice and reacted secondary antibody. For nucleus detection, it stained with 1% Hoechst33342 for 20 min. Fluorescence was detected by confocal (Carl Zeiss, Thornwood, N.Y., USA).

Results

The inhibitory effect of TGF-β-dependent signaling pathway on cancer cell metastasis was confirmed in TAN environment by PLAG treatment. As a result, it was confirmed that the increase of SMAD activity, which is an increased TGF-βsignal pathway by TAN and G-CSF co-stimulation, decreased by PLAG treatment. Also, we confirmed that the formation of the SMAD2/3-SMAD4 complex for nucleus translocation decreases with PLAG treatment. We confirmed that the nucleus translocation of SMAD2/3 and SMAD4 increased by TAN and G-CSF co-stimulation decreased with PLAG treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for treating a subject suffering from cancer, comprising:
administering to the subject:
a) a granulocyte colony-stimulating factor (G-CSF) compound; and
b) a compound of Formula (I): 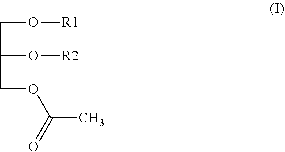
wherein R1 and R2 are independently a fatty acid group comprising 14 to 20 carbon atoms.

2. A method of reducing tumor volume in a subject having a solid tumor, comprising:
administering to a subject having a tumor an effective amount of a compound of Formula (I): 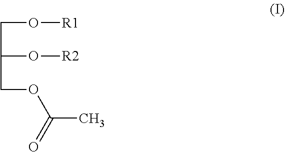
wherein R1 and R2 are independently a fatty acid group comprising 14 to 20 carbon atoms,
and wherein volume of the tumor is reduced.

3. The method of claim 1, wherein the compound of Formula (I) is a compound of Formula II:

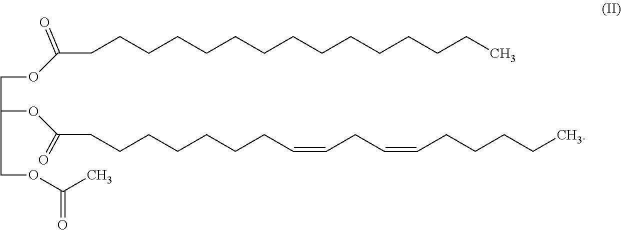 [SMILES: CCCCC/C=C\C/C=C\CCCCCCCC(=O)OC(COC(C)=O)COC(=O)CCCCCCCCCCCCCCC]

4. The method of claim 1 wherein the subject has been previously treated with a G-CSF compound but without a compound of Formula (I).

5. The method of claim 2 wherein a granulocyte colony-stimulating factor (G-CSF) compound is also administered to the subject.

6. The method of claim 1 further comprising administering c) a chemotherapeutic agent distinct from the a) G-CSF compound and the b) compound of Formula (I).

7. The method of claim 6 wherein the c) chemotherapeutic agent is selected from cyclophosphamide, doxorubicin, etoposide, ifosfamide, mesna, cisplatin, gemcitabine and/or tamoxifen.

8. A kit for the treatment of cancer comprising:
a) a granulocyte colony-stimulating factor (G-CSF) compound; and
b) a compound of Formula (I): 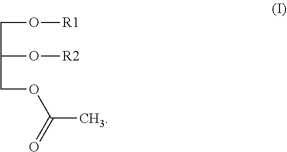
wherein R1 and R2 are independently a fatty acid group comprising 14 to 20 carbon atoms.

9. The kit of claim 8 further comprising directions for the treatment of cancer.

10. The method of claim 2 wherein the subject has been previously treated with a G-CSF compound but without a compound of Formula (I).

11. The method of claim 2 wherein the subject has been previously treated with a G-CSF compound but without a compound of Formula (I).

12. The method of claim 3 further comprising administering c) a chemotherapeutic agent distinct from the a) G-CSF compound and the b) compound of Formula (I).

13. The method of claim 2 further comprising administering c) a chemotherapeutic agent distinct from the a) G-CSF compound and the b) compound of Formula (II).


SEQUENCE LISTING (8 sequences)

SEQ ID NO: 1 - DNA, 20 bp, Artificial Sequence
  forward primer
gacaatgcccctcaagtgtt

SEQ ID NO: 2 - DNA, 20 bp, Artificial Sequence
  reverse primer
ccattaagccgagtgatggt

SEQ ID NO: 3 - DNA, 19 bp, Artificial Sequence
  forward primer
gagaactttgccgttgaag

SEQ ID NO: 4 - DNA, 20 bp, Artificial Sequence
  reverse primer
tccagcagcttcctgtaggt

SEQ ID NO: 5 - DNA, 20 bp, Artificial Sequence
  forward primer
ccccaatcggaagcctaact

SEQ ID NO: 6 - DNA, 19 bp, Artificial Sequence
  reverse primer
acagagtcccagatgagca

SEQ ID NO: 7 - DNA, 23 bp, Artificial Sequence
  forward primer
caaggtcatccatgacaactttg

SEQ ID NO: 8 - DNA, 22 bp, Artificial Sequence
  reverse primer
gtccaccaccctgttgctgtag